(19) United States
(12) Patent Application Publication
(10) Pub. No.: US 2004/0153154 A1
(43) Pub. Date: Aug. 5, 2004

(54) Bone implant and method for manufacturing same

(76) Inventor: Wolfgang Dinkelacker, Sindelfingen (DE)

Correspondence Address: KUDIRKA & JOBSE, LLP, ONE STATE STREET, SUITE 800, BOSTON, MA, 02109, US

(21) Appl. No.: 10/762,809

(22) Filed: Jan. 22, 2004

(30) Foreign Application Priority Data
Jan. 23, 2003 (EP) 03 001 516.8

(51) Int. Cl. A61F 2/28

(52) U.S. Cl. 623/16.11

(57) ABSTRACT

Parts of the surface of a bone implant that come in contact with bone cells are pretreated by sandblasting and/or acid etching and the pretreated microstructure is reshaped to produce a microstructure with an array of densely packed rounded domes separated by rounded lacunae. The size of the domes, the spacing between the domes and the depth of the lacunae are the same order of magnitude as the average size of the bone cells and the cell projections connecting the bone cells. The reshaping may be accomplished by sputtering a cover layer consisting of a biocompatible material onto the pretreated surface parts. A nanostructure comprised of an array of densely packed rounded domes separated by rounded lacunae can further superimposed on the microstructure of the implant surface.

### FIELD OF THE INVENTION

This invention relates to a bone implant having a surface that is compatible with bone cells to achieve a secure anchoring of the implant in human bone. This invention also relates to a method of manufacturing an implant surface that is compatible with bone cells.

### BACKGROUND OF THE INVENTION

Known bone implants are made of a material having a good compatibility with the tissue of human bone. Titanium and titanium alloys as well as tantalum and tantalum alloys are especially suitable. To promote rapid and permanent integration of the implant in the bone, the known implants are provided with a surface roughness in the areas where they come in contact with the bone, thus improving contact of the bone cells with the implant surface and increasing the resistance of the integrated implant to lateral forces. The type of surface roughness and its dimensions are of crucial importance for secure and long-lasting anchoring of the implant in the bone. A distinction is made between a “macroroughness” in the form of structures like a screw thread, ribs, grooves, ridges or gratings and a “microroughness” in the form of pores, pits, depressions or lacunae that are very small in comparison with the macroroughness structures (see, for example, European Patent EP-A 1013236). The roughness values in the range of the macroroughness are between 10 and 150 μμm and those in the range of microroughness are between 0.5 and 3 μm.

Extensive research has been conducted to obtain an implant surface with biocompatible properties (for example, D. Buser et al, “Influence of Surface Characteristics on Bone Integration of Titanium Implants,” Journal of Biomedical Materials Research, Vol. 25, pp. 889-902, John Wiley & Sons, Inc., 1991). The contact with the surrounding bone tissue is facilitated by treating the surface of the implant body, and the implant surface involved in the contact is to be enlarged. It is also known that the surface of the implant body can be exposed to an acid etching process (after removal of the natural titanium oxide layer) to achieve an essentially uniform roughness over the entire surface (WO 96/16611). In addition, surface coatings with materials having a good compatibility with bone, e.g., a coating with hydroxyl apatite (German Patent DE-A 38 39 724) or with mother of pearl powder (French Patent FR-A 2715568).

Furthermore, there are also known titanium plasma spray (TPS) coatings, which are produced by thermal spraying of titanium onto a titanium implant. According to another known method, first the surface is treated by coarse sandblasting with corundum, which produces a macroroughness on the titanium, which produces a macroroughness on the titanium. This process is followed by an acid etching which produces micropitting distributed over the sandblasted surface (Cochran et al, “Bone Response to Unloaded and Loaded Titanium Implants with a Sandblasted and Acid-Etched Surface,” Journal of Biomedical Materials Research, vol. 40, 1998, p. 1; European Patent EP-A 0388567).

It is also known that the surface of metal implants can be treated by ion beam sputtering to improve their biocompatibility (A. J. Weigand et al, “Ion-Beam-Sputter Modification of the Surface Morphology of Biological Implants,” Journal Vacuum Society Technology, vol. 14, no. 1, January/February 1977, pp. 326-331).

In some of the known methods, the lack of hardness and adhesiveness of the surface coating may be a problem. In other methods, impurities are created in the surface of the implant and then must be removed by complex method steps. One example of this is the treatment of the implant surface by sandblasting, which leaves inclusions of the blasting particles on the implant surface. These inclusions may make it difficult for the implant to become integrated and must therefore be removed subsequently by acid etching. It is a particular disadvantage that sandblasting and treatment of the implant surface with acid leave behind a surface structure which has sharp edges with pointed jags and peaks and indentations tapering to a point. The biocompatibility of a surface with sharp-edged fissures is limited because of inadequate adaptation to the shape of the bone cells adjacent to the implant and their cell projections.

The goal of this invention is to produce a bone implant surface that will have an improved biocompatibility while still having sufficient hardness and adhesiveness and avoiding the aforementioned disadvantages.

### SUMMARY OF THE INVENTION

In accordance with the principles of the present invention, a bone implant has a macrostructure on which is superimposed a microstructure that comprises an array of densely packed rounded elevations (hereinafter called “domes”) separated by rounded lacunae. The size of the domes, their distance from one another and the depth of the lacunae are substantially the same order of magnitude as the average size of the bone cells and the cell projections connecting them. Such an implant has the advantage that the interface with which the bone tissue comes into contact has a topography which corresponds largely to the shape of the bone cells, which have only rounded structures without edges. This correspondence facilitates adaptation of the cells to the interface and their migration when the implant is under load.

According to a preferred embodiment, the implant body is fabricated from titanium or a titanium alloy and the microstructure is formed by a cover layer which is on a part of the implant body surface that has been pretreated by sandblasting and/or acid etching. The cover layer may be a sputtered or electrodeposited layer, preferably consisting of titanium or a titanium alloy.

Also in accordance with the principles of the invention, a method is described for producing a bone implant, in particular, a dental implant, which has an implant body preferably made of titanium or a titanium alloy having a biocompatible surface. This surface is roughened by pretreatment, such as sandblasting or acid etching or by sandblasting with a subsequent acid etching. The pretreated surface is then modified by application or removal of material to produce an array of densely packed rounded domes separated by rounded lacunae. The size of the domes, their distances from one another and the depth of the lacunae are substantially the same order of magnitude as the average size of the bone cells.

According to another aspect of this invention, the modification of the pretreated surface may be accomplished by applying a cover layer of titanium or a titanium alloy, which is applied by sputtering or electrodeposition, preferably by sputtering. One advantage of this method is that impurities which become incorporated into the surface of the implant during the pretreatment stage are permanently enclosed by the cover layer. The cover layer applied by sputtering has a high hardness and good adhesion to the implant surface. The points and sharp-edged indentations in the microstructure of the implant surface formed by sandblasting and/or acid etching are rounded off and equalized by the cover layer. This prevents cell damage in contact of the bone cells with the implant surface.

According to another advantageous embodiment of this invention, an additional nanostructure is superimposed on the microstructure formed by the rounded domes and rounded lacunae between them. This nanostructure is also formed of rounded elevations or domes separated by rounded lacunae. However, the size of the nanostructure domes, the distances between the nanostructure domes, and the nanostructure lacunae depth are smaller by a decimal order of magnitude than the corresponding dimensions of the microstructure. The nanostructure offers points of attack for the cell projections and thus facilitates the migration of the bone cells.

### BRIEF DESCRIPTION OF THE DRAWINGS

The above and further advantages of the invention may be better understood by referring to the following description in conjunction with the accompanying drawings in which:

FIG. 1 is a greatly enlarged cross-sectional diagram of a part of the surface of a bone implant according to the state of the art;

FIG. 2 is a schematic diagram of two bone cells in contact with a part of a known implant surface;

FIG. 3 is a greatly enlarged partial section of a first exemplary embodiment of the surface of a bone implant according to FIG. 1 after it has been reshaped according to this invention;

FIG. 4 is an enlarged schematic diagram of several bone cells in contact with a part of an implant surface according to this invention;

FIG. 5 is a greatly enlarged cross-sectional diagram of a part of another exemplary embodiment of the bone implant produced according to this invention with a pretreated surface and a cover layer;

FIG. 6 is a schematic view of a cover layer produced by sputtering, shown in a three-dimensional diagram;

FIG. 7 is an enlarged cross-sectional diagram of part of an implant surface having a macrostructure in the form of groove-like recesses and a microstructure covering the macrostructure according to this invention; and

FIG. 8 is an enlarged cross-sectional diagram of part of an implant surface having an inventive microstructure and a nanostructure covering the microstructure.

### DETAILED DESCRIPTION

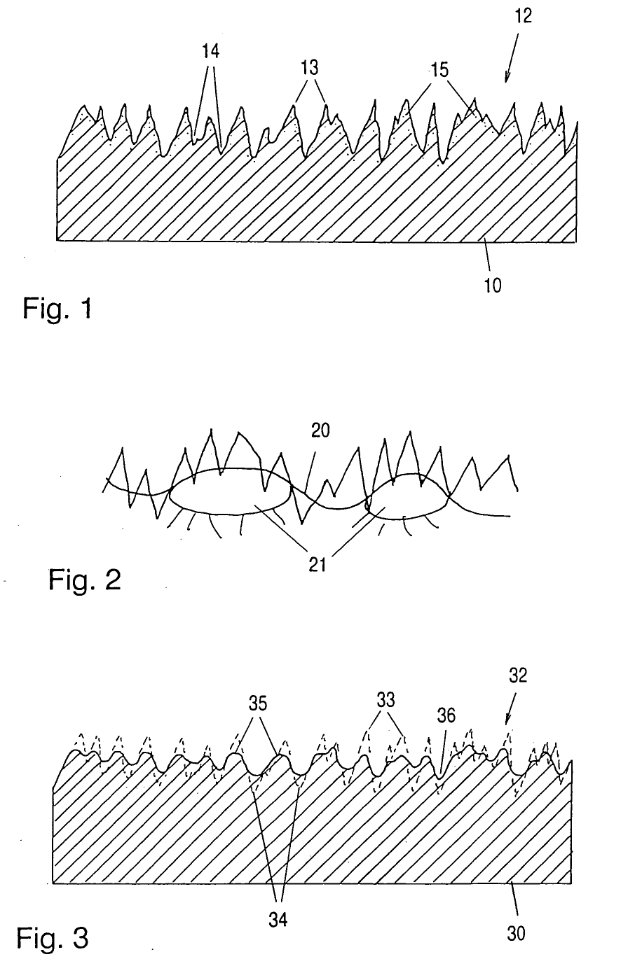

The cross-sectional diagram in FIG. 1 shows a detail of the surface of a known bone implant 10, which surface 12 has been treated by sandblasting with corundum or aluminum oxide in a known way. This sandblasting produces a microstructure consisting of a plurality of jags and peaks 13 with sharp-edged notch-like recesses 14 between them. This structure represents an inadequate adaptation to the structure of the bony substance in the cell area (FIG. 2). Cell damage may occur when cells 21 come in contact with the implant 20, thereby delaying integration of the implant in the bone and possibly even leading to loss of the implant. Furthermore, particles 15 of the sandblasting material may remain in the surface 12; these are impurities of the bone-compatible implant material and may cause local inflammation, which has a negative effect on the healing process with the implant.

The cross-sectional diagram in FIG. 3 shows a corresponding detail from the surface of a bone implant 30 in which the microstructure has been reshaped according to this invention. The bone implant 30 may be a dental implant or an implant for replacement of a hip joint or a knee joint or some other bone implant suitable for use in the human body. The implant 30 is made of a material which is very compatible with the bony substance of the human body and also has the required hardness. This material is preferably titanium or a titanium alloy. Instead, in certain cases, tantalum or tantalum alloys or ceramic materials may also be used.

The surface 32 is pretreated by sandblasting with corundum or aluminum oxide to produce a surface structure having a plurality of sharp-edged jags and points 33 and between them sharp-edged notch-like indentations 34, corresponding to the jagged surface structure 13, 14 shown in FIG. 1. In another method step, the surface that has been roughened by the pretreatment is reshaped by applying or removing material so that the jags and peaks 33 are worn away and the notch-like indentations 34 are filled up to some extent. The result of this reshaping is a plurality of densely packed, rounded domes 35 that are separated by rounded lacunae 36 with an average peak-to-valley height in the range of 1-10 μm, preferably 1.5 μm and a diameter in the range of 1.5-10 μm, preferably 5 μm.

The peaks and jags 33 are preferably rounded by the action of a high-power laser beam, which is guided over the surface. The laser beam creates rounded domes 35 at the site of the protruding peaks and jags 33 due to vaporization of material and rounded lacunae 36 at the site of the notch-like indentations 34 due to displacement of material. A surface structure reshaped in this way has an improved compatibility with the structure of bone tissue.

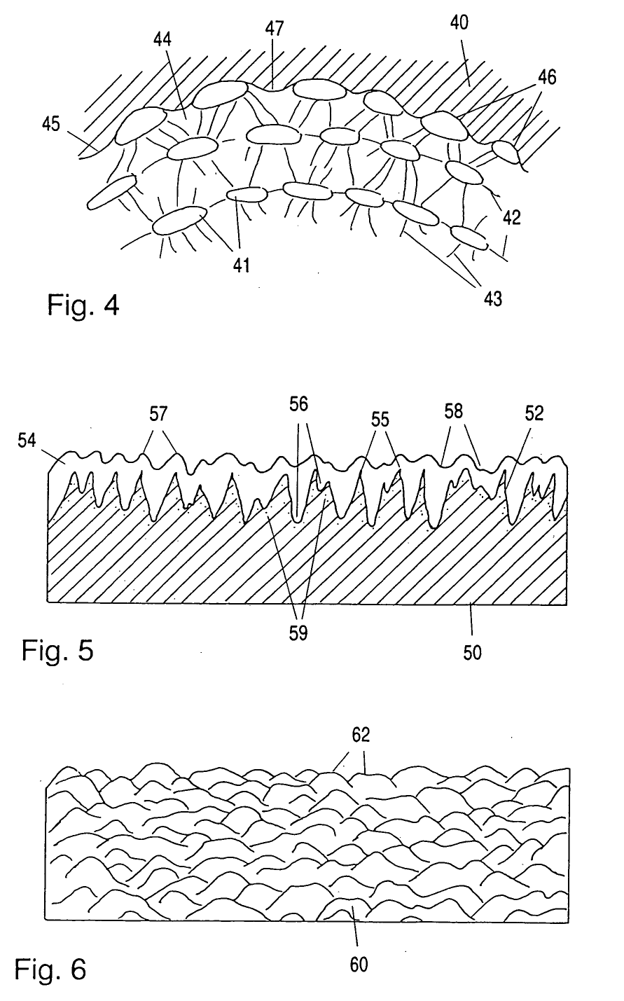

The bone tissue that comes in contact with the implant after the surgery consists of bone cells and cell projections, in particular osteoblasts, and the osteocyte formed from the former, joining the osteocytes together. The osteocytes are almond-shaped and have a length of approximately 3-10 μm and a thickness of approximately 3 μm. The parameters of the pretreatment (particle size of the sandblasting material) and the reshaping operations (the power of the laser beam and the speed of the scanning movement) are selected so that the size of the domes 35 and their distance from one another as well as the depth of the lacunae 36 have the same order of magnitude as the average size of the osteocytes and the cell projections connecting them. The bone cells that are formed at the interface 45 with the implant 40 after the surgery thus encounter a structure which is adapted to their size and shape and facilitates their contact with the implant. This is illustrated in the simplified and schematic diagram in FIG. 4. Almond-shaped osteocytes 41 are arranged along the bone lamellae 42 and are joined together by cell projections 43. The unstructured matrix 44 of the bone tissue is located between these cell structures.

The process of bone cells coming in contact with the surface of the implant is dynamic. In this reorientation, the cells 41 migrate on the interface 45 under the influence of their cell projections 43. In doing so, cells 41 become deposited in the lacunae 46 of the surface of the implant. The rounded domes 47 facilitate the cell movements and reduce the risk of cell damage occurring here. A similar effect also occurs on the surface of the implant that has become integrated when it is placed under load in the patient's jaw. Here again, there is movement of the bone cells in the area of the interface with the implant. Osteocytes that are no longer functional are degraded here under the influence of osteoclasts and newly differentiated osteoblasts as well as osteocytes that have been preserved and the amorphous bony substance surrounding them migrate into lacunae 46 that have become free. These processes are facilitated by the rounded structures 46, 47 or are made possible for the first time through these structures.

As an alternative to the use of lasers described above, a galvanic treatment may also be used to round off the peaks and jags 33 and to round off the notch-like indentations 34. To remove the roughness peaks, an electrode arrangement is connected so that the implant surface functions as the cathode. An erosion of parts of the walls of the sharp-edged indentations also occurs here so that they are reshaped to rounded lacunae.

The reshaping of the pretreated implant surface to form a microstructure consisting of rounded elevations or domes on rounded lacunae may also be accomplished by applying a cover layer. To do so, in another method step, a cover layer 54 which produces a smoothing of the surface of the implant 50 is applied preferably by sputtering to the surface 52 of an implant 50 pretreated by sandblasting and/or acid etching (FIG. 5).

Sputtering, also known as ion sputtering or cathode atomization, is a method that is known per se and is used for, among other things, producing thin coatings on metallic or nonmetallic surfaces; this method has already been proposed for coating bone implants. In this regard, reference is made to the publication by A. J. Weigand et al, “Ion-Beam-Sputter Modification of the Surface Morphology of Biological Implants,” Journal Vacuum Society Technology, vol. 14, no. 1, January/February 1977, pages 326-331. Metal layers produced by sputtering have a high hardness and a good and long-lasting adhesion to the coated substrate.

The starting material for the sputtering step is preferably titanium or a titanium alloy that is applied to the surface of the implant in the presence of a protective gas, such as argon. The thickness of the sputtered cover layer 54 depends on the duration of the sputtering process and the number of sputtering runs and is between a few tenths of a micrometer and a few micrometers, preferably between 0.1 μm and 2 μm, e.g., 0.5 μm. Depending on the type of implant, the cover layer is sputtered to yield a thickness adapted approximately to the surface roughness of the bone cell profile.

The cover layer 54 is designed to cover the peaks and jags 55 produced by sandblasting and/or acid etching and partially fills up the sharp-edged notch-like indentations 56 between these peaks. This produces a wavy microstructure on the surface of the implant, which reshapes the peaks and jags 55 into rounded domes 57 and reshapes the sharp-edged deep areas 56 into rounded lacunae 58. FIG. 6 shows an example of a surface 60 with rounded domes 62 produced by sputtering. The cover layer 54 has a thickness which corresponds approximately to the surface roughness of the cell profile. It may have a thickness between 0.1 and 2 μm, for example.

The surface structure produced by the cover layer 54 has the same advantages as those explained above with reference to FIGS. 3 and 4. Another effect is that residues of sandblasting material and other impurities which might become embedded in the surface 52 in sandblasting are covered and sealed by the cover layer 54. The impurities 59 are thus isolated from the bony substance and cannot cause inflammation or otherwise interfere with the integration of the implant.

As an alternative to application by sputtering, the cover layer 54 may also be produced by a galvanic deposition which is deposited over the roughness peaks of the pretreated surface of the implant and rounds them while at the same time partially filling up the sharp-edged indentations so that they are reshaped to yield rounded lacunae.

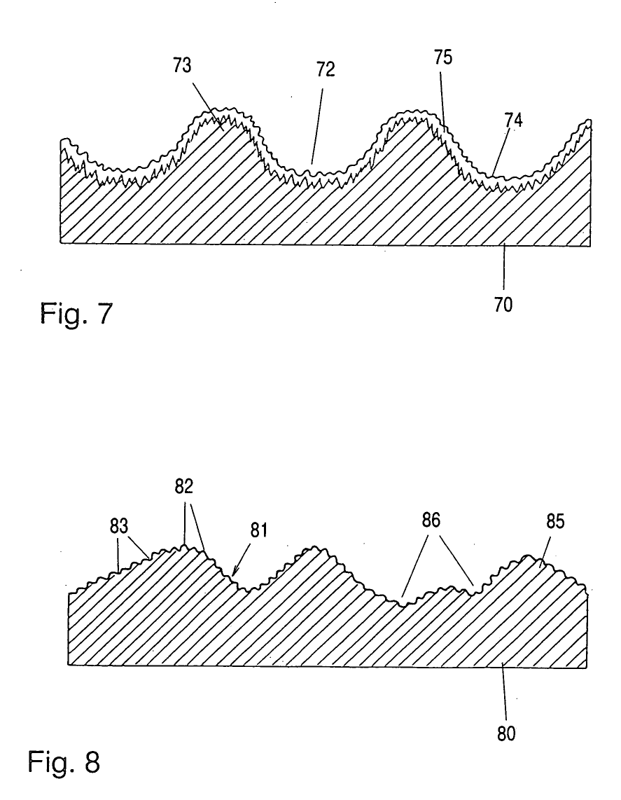

The method according to this invention is preferably used with bone implants whose surfaces have a macrostructure. This is a structure suitable for applying load to the implant and being anchored permanently in the bone such as screws, self-tapping screws, axial or diagonal fluting, grooves or ridges. The cross-sectional diagram in FIG. 7 shows a detail of the surface of a maxillary implant 70, the surface of which has a macrostructure in the form of groove-like indentations 72 and ridges 73 delineating the latter of the type described in European Patent EP-A 1013236 A. The indentations 72 are of an order of magnitude which corresponds to the order of magnitude of the osteons of the bone tissue which are deposited on the groove-like indentations. The macrostructure formed by the groove-like indentations 72 and the ridges 73 is sandblasted and/or acid etched so that a uniform roughness is created over the entire surface of the indentations 72 and ridges 73, corresponding to the microstructure of the surface 12 in FIG. 1. A cover layer 75 is applied to the surface treated in this way by sputtering or by galvanic deposition, covering the groove-like indentations. This results in a cell-friendly surface, which is biologically compatible to a high degree in the sense of effective cell integration.

In another optional method step, a nanostructure may be superimposed on the microstructure designed according to this invention so that the form of the nanostructure corresponds to that of the microstructure described here and also has rounded domes and rounded lacunae. The dimensions of the domes, their spacing and the depth of the lacunae in the nanostructure are smaller by approximately one decimal order of magnitude than those of the microstructure described here. The depth of the lacunae of the nanostructure may be in the range of 10-500 nm, e.g., 250 nm, and the distance between the elevations may be in the range of 100 to 500 nm, e.g., 250 nm. FIG. 8 shows an example of a nanostructure 81 comprising a plurality of small domes 82 and lacunae 83 applied to the domes 85 and lacunae 86 of the inventive microstructure in an implant 80. The domes 85 and lacunae 86 correspond in shape and arrangement to the domes 35 and lacunae 36 of the microstructure. The nanostructure is produced by another method step on an implant surface reshaped according to this invention. This is preferably accomplished by sputtering, whereby the parameters of the sputtering run are adjusted accordingly to produce the fine structure. As an alternative, the nanostructure may be produced by sputtering by using a finely focused laser beam which is modulated according to the structure to be produced. The nanostructure has the effect that the cell projections extend into the lacunae, where they can be deposited, so that their function in cell migration is supported.

In the production of the microstructure, the pretreatment by sandblasting and/or acid etching and the subsequent reshaping according to this invention, including the application of a cover layer are limited to the parts of the surface of the implant which come in contact with the bony substance after the implant has been implanted. To do so, the surface of the implant is masked so that the parts of the surface to be treated are delineated from the other parts of the surface of the implant, with the other parts of the surface being covered during the pretreatment and the reshaping.

Although this invention is described here on the basis of a preferred embodiment, modifications and other embodiments may be implemented without going beyond the scope of this invention as defined by the claims.

What is claimed is:

1. In a bone implant for a bony substance having an implant body with a surface that is compatible with bone cells having an average size and wherein the surface has a macrostructure that contacts the bony substance and a microstructure for anchoring the implant in the cell area, the improvement wherein the microstructure comprises an array of densely packed rounded domes separated by rounded lacunae, and wherein the size of the domes, their distances from one another and the depth of the lacunae are substantially the same order of magnitude as the average size of the bone cells.

2. The improvement of claim 1, wherein parts of the implant body surface are pretreated by sandblasting and acid etching and wherein the microstructure comprises a cover layer formed on the implant body surface parts.

3. The improvement of claim 2 wherein the cover layer is fabricated from a material in the group consisting of titanium and titanium alloys.

4. The improvement of claim 2 wherein the bone cells have a profile with a surface roughness and wherein the cover layer comprises a layer of sputtered material having a thickness which corresponds substantially to the surface roughness of the bone cell profile.

5. The improvement of one of claims 2-4 wherein the cover layer has a thickness between 0.1 and 2 micrometers.

6. The improvement of one of claims 2-4, wherein implant body surface parts which are pretreated by sandblasting and acid etching and on which is formed the cover layer are delineated from other surface parts of the implant by masking.

7. The improvement of claim 1 further comprising a nanostructure superimposed on the microstructure, the nanostructure being comprised of a densely packed array of rounded domes separated by rounded lacunae, wherein the size of the nanostructure domes, the distances from one nanostructure dome to another and the depth of the nanostructure lacunae are smaller than the corresponding dimensions of the microstructure by approximately one decimal order of magnitude.

8. The improvement of claim 7, wherein the depth of the nanostructure lacunae is in the range of 10-500 nm wherein and the distance between nanostructure domes is in the range of 100-500 nm.

9. A method of producing a bone implant that is compatible with bone cells having an average size, the method comprising:
(a) fabricating an implant body with a biocompatible surface,
(b) pretreating parts of the implant body surface to roughen the implant body surface;
(c) reshaping the roughened implant body surface by application and removal of material to create a microstructure comprised of an array of densely packed rounded domes separated by rounded lacunae; and
(d) selecting parameters of steps (b) and (c) so that a size of the domes, distances from one dome to another and a depth of the lacunae are substantially the same order of magnitude as the average size of the bone cells.

10. The method of claim 9 wherein step (b) comprises sandblasting the implant body surface.

11. The method of claim 9 wherein step (b) comprises acid etching the implant body surface.

12. The method of claim 9 wherein step (b) comprises sandblasting the implant body surface and acid etching the sandblasted implant body surface.

13. The method of any one of claims 8-12, wherein step (c) comprises applying a cover layer to the roughened implant body surface.

14. The method of claim 13, wherein step (c) comprises applying the cover layer by sputtering.

15. The method of claim 13, wherein step (c) comprises applying the cover layer by electroplating.

16. The method of any one of claims 9-12, wherein step (c) comprises treating the roughened implant body surface with a laser so that roughness peaks produced by step (b) are worn down and notch-like indentations are reshaped to form rounded lacunae.

17. The method of any one of claims 9-12, wherein step (c) comprises treating the roughened implant body surface by a galvanic erosion process so that roughness peaks produced by step (b) are worn down and sharp-edged indentations are filled up to form rounded lacunae.

18. The method according to claim 17, wherein the roughened implant body surface functions as a cathode in the galvanic erosion process for removal of the roughness peaks.

19. The method of claim 15, wherein the cover layer is applied up to a thickness that corresponds substantially to a surface roughness of a bone cell profile.

20. The method of claim 19, wherein the cover layer is applied to a thickness between 0.1 and 2 micrometers.

21. The method of claim 9, wherein the implant body surface parts to be treated in step (b) and step (c) are delineated from other surface parts of the implant body by masking, and the other surface parts are covered while the method steps (b) and (c) are being carried out.

22. The method of claim 9, wherein step (a) comprises fabricating an implant body with a macrostructure to fasten the implant body into bone and wherein the microstructure formed by steps (b) and (c) is applied to the macrostructure.

23. The method of claim 9, further comprising:
(e) forming a nanostructure on the microstructure, the nanostructure being formed by an array of rounded domes separated by rounded lacunae wherein a size of the nanostructure domes, a spacing between the nanostructure domes and a depth of the nanostructure lacunae are substantially one decimal order of magnitude smaller than corresponding dimensions of the microstructure.

24. The method of claim 23, wherein the depth of the nanostructure lacunae is in the range of 10-500 nm and the spacing between the nanostructure domes is in the range of 100-500 nm.
